(19) United States
(12) Patent Application Publication
(10) Pub. No.: US 2022/0211725 A1
(43) Pub. Date: Jul. 7, 2022

(54) Pharmaceutical Composition Comprising Venetoclax

(71) Applicant: Alembic Pharmaceuticals Limited, Vadodara (IN)

(72) Inventors: Pankaj Kumar Khapra, Vadodara (IN); Rahul Dabre, Hyderabad (IN); Prasanna Barada Sahoo, Hyderabad (IN); Murthy V.S.N. Dwibhashyam, Hyderabad (IN)

(73) Assignee: Alembic Pharmaceuticals Limited, Vadodara (IN)

(21) Appl. No.: 17/606,925

(22) PCT Filed: May 6, 2020
(86) PCT No.: PCT/IB2020/054273

§ 371 Date: Oct. 27, 2021

(30) Foreign Application Priority Data
May 7, 2019 (IN) 201921018227

(51) Int. Cl. A61K 31/635; A61K 9/20

(52) U.S. Cl. CPC A61K 31/635; A61K 9/2027; A61K 9/2018; A61K 9/2009; A61K 9/2095

(57) ABSTRACT

The present subject matter provides amorphous solid dispersions of venetoclax or a pharmaceutically acceptable salt thereof, and pharmaceutical compositions comprising said amorphous solid dispersions. The present subject matter also provides methods for the preparation of said solid dispersions and compositions. The present subject matter further provides pharmaceutical compositions comprising mixture of solid dispersions.

### CROSS-REFERENCE TO RELATED APPLICATIONS

This application is the National Stage entry of PCT/IB2020/054273, filed on May 6, 2020, which claims priority to India Patent Application Number IN2019210182275 filed on May 7, 2019, both of which are incorporated herein by reference in their entireties.

### FIELD OF THE INVENTION

The field of the invention relates to pharmaceutical compositions comprising venetoclax or a pharmaceutically acceptable salt thereof and process for their preparation.

### BACKGROUND OF THE SUBJECT MATTER

Venetoclax is a selective inhibitor of BCL-2 protein chemically designated as 4-(4-{[2-(4-chlorophenyl)-4,4dimethylcyclohex-1-en-1-yl]methyl}piperazin-1-yl)-N-({3-nitro-4-[(tetrahydro-2H-pyran-4ylmethyl)amino]phenyl}sulfonyl)-2-(1H-pyrrolo[2,3-b]pyridin-5-yloxy)benzamide) corresponding to the molecular formula C45H50ClN7O7S, molecular weight of 868.44 and the following structure:

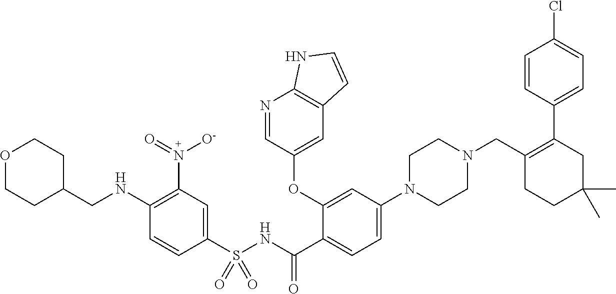

In the United States, venetoclax is available as 10 mg, 50 mg and 100 mg film-coated tablets under the brand name VENCLEXTA®. Venetoclax exists as light yellow to dark yellow solid, with very low aqueous solubility and limited bioavailability because of which it may be classified as BCS Class II/IV compound.

For a poorly soluble drug, one of the main challenges is to increase its solubility/bioavailability through formulation. Solid dispersion is one of these methods, which is applied to improve the solubility, dissolution rates and consequently, the bioavailability of poorly soluble drugs. The solid dispersion is based on the concept that the drug is dispersed in an inert carrier at solid state. Quite often, the drug in solid dispersion is in an amorphous state, which has increased solubility compared to the crystalline state. However, one of the main challenges in utilization of solid dispersion is to maintain the amorphous state of the drug and avoid crystallization, as this may negate the solubility/bioavailability improvements achievable by the amorphous state. Another challenge in utilization of solid dispersion is precipitation of amorphous drug to crystalline state in the gastrointestinal tract, which may again lead to decreased or non-reproducible solubility/bioavailability.

Advancements in solid dispersion science have led to some solutions to the aforementioned challenges. For example, a review by Vasconcelos et al., entitled “Solid dispersions as strategy to improve oral bioavailability of poor water soluble drugs,” Drug Discovery Today, Volume 12, Numbers 23/24, December 2007, reports a solid dispersion system termed “Third generation solid dispersions,” which contains surfactant as an additional ingredient apart from the carrier/polymer and poorly soluble drug. It is reported that the inclusion of surfactants in solid dispersion containing a polymeric carrier may help to prevent precipitation and/or formation of larger hydrophobic particles by agglomeration of fine crystalline precipitate. Vasconcelos further reports that drug recrystallization is a major barrier to the marketing success of conventional solid dispersions, and this could be prevented by usage of third generation solid dispersion strategies.

However, presence of surfactant inside solid dispersion containing drugs brings its own challenges, such as processing and manufacturability. U.S. Patent Publication No. US 2015/0148331 discloses solid dispersion comprising venetoclax, water soluble polymer and surfactant prepared by hot melt extrusion technique. This publication emphasizes the presence of surfactant in the solid dispersion along with drug and hydrophilic polymer. However, it also teaches that although higher surfactant level leads to faster drug release rate, greater surfactant level may affect the manufacturability of the extrudates in the preparation of solid dispersions.

U.S. Pat. No. 10,213,433 discloses the preparation of solid dispersion comprising venetoclax, water soluble polymer and surfactant by solvent evaporation technique.

WO 2017/212431 discloses various polymorphic forms of venetoclax and processes of preparing them. It also discloses solid dispersions of venetoclax prepared generally by solvent evaporation technique. It does mention hot-melt extrusion to prepare solid dispersion but does not provide any information regarding such processes. The examples are provided with only solvent evaporation technique.

Formulators often face challenges with solid dispersions such as re-crystallization, precipitation of drug, instability and lack of clarity i.e. opaqueness. Hence, there remains a need of a solid dispersion comprising venetoclax that is devoid of one or more of such disadvantages.

In addition, hot melt extrusion process to prepare a solid dispersion is often constrained by manufacturability and scalability challenges. Such issues in many cases depend on the composition mix that is to be processed. Therefore, there is a need of a solid dispersion comprising venetoclax that is amenable to an improved, faster process which is continuous, uses less energy and is economical.

During the course of formulation development, it was found that the difficulties associated with a composition comprising a solid dispersion of venetoclax may be overcome by preparing a composition comprising either (i) a solid dispersion comprising venetoclax or a pharmaceutically acceptable salt thereof, at least two different water soluble polymer and free of a surfactant, or (ii) a mixture of at least two solid dispersions, i.e., a solid dispersion comprising venetoclax or a pharmaceutically acceptable salt thereof and a water soluble polymer and free of a surfactant, and a solid dispersion free of venetoclax or a pharmaceutically acceptable salt thereof comprising a water soluble polymer and a surfactant. These compositions may have one or more or all of following advantages:
1) Improved dissolution,
2) Improved chemical stability (for example, reduced relative substances),
3) Improved stability of amorphous venetoclax (for example, less crystallinity),
4) Improved manufacturability,
5) Bio-equivalency with VENCLEXTA®, which contains a solid dispersion component containing venetoclax, a water soluble polymer and a surfactant.

### SUMMARY OF THE SUBJECT MATTER

An object of the present subject matter is to provide a solid dispersion comprising venetoclax or a pharmaceutically acceptable salt thereof and one or more polymers.

An object of the present subject matter is to provide a solid dispersion comprising venetoclax or a pharmaceutically acceptable salt thereof and at least two polymers.

An object of the present subject matter is to provide a stable solid dispersion comprising venetoclax or a pharmaceutically acceptable salt thereof, wherein amorphous state of venetoclax or a pharmaceutically acceptable salt thereof is retained during stability.

An object of the present subject matter is to provide a stable solid dispersion comprising venetoclax or a pharmaceutically acceptable salt thereof, wherein amorphous venetoclax or a pharmaceutically acceptable salt thereof does not exhibit significant crystallinity.

An object of the present subject matter is to provide a stable solid dispersion comprising venetoclax or a pharmaceutically acceptable salt thereof, wherein venetoclax or a pharmaceutically acceptable salt thereof does not undergo significant degradation.

An object of the present subject matter is to provide a solid dispersion comprising venetoclax or a pharmaceutically acceptable salt thereof, wherein the solid dispersion is prepared by hot melt extrusion with improved manufacturability.

An object of the present subject matter is to provide a pharmaceutical composition comprising a solid dispersion comprising venetoclax or a pharmaceutically acceptable salt thereof and one or more polymers.

An object of the present subject matter is to provide a pharmaceutical composition comprising a solid dispersion comprising venetoclax or a pharmaceutically acceptable salt thereof and at least two polymers.

An object of the present subject matter is to provide a pharmaceutical composition comprising a solid dispersion comprising venetoclax or a pharmaceutically acceptable salt thereof and at least two polymers, wherein the pharmaceutical composition is bioequivalent to VENCLEXTA®.

In an embodiment, the solid dispersion comprising venetoclax disclosed herein may be devoid of a surfactant.

In an embodiment, the solid dispersion comprising venetoclax disclosed herein may comprise a surfactant.

In an embodiment, the solid dispersion disclosed herein is prepared using amorphous form of venetoclax or a pharmaceutically acceptable salt thereof.

In an embodiment, the subject matter provides a solid dispersion comprising venetoclax or a pharmaceutically acceptable salt thereof, a first polymer, a second polymer, and optionally one or more pharmaceutically acceptable excipients.

In an embodiment, the subject matter provides a solid dispersion comprising venetoclax or a pharmaceutically acceptable salt thereof, a first polymer, a second polymer, and optionally one or more pharmaceutically acceptable excipients, wherein the solid dispersion is devoid of any surfactant.

In an embodiment, the first polymer and the second polymer are not the same.

In an embodiment, first and second polymers may be selected from hydrophilic water soluble polymers, enteric coating polymers and water insoluble polymers.

In an embodiment, the first polymer is a hydrophilic water soluble polymer and the second polymer is an enteric coating polymer.

In an embodiment, the first polymer is an enteric coating polymer and the second polymer is a hydrophilic water soluble polymer.

In an embodiment, both first polymer and second polymers are hydrophilic water soluble polymers.

In an embodiment, the second water soluble polymer is a hydrophilic water soluble polymer other than first water soluble polymer.

In an embodiment, the first polymer is a vinyl based polymer and second polymer is a cellulose derivative.

In an embodiment, the first polymer is co-povidone or povidone and second polymer is hydroxypropyl cellulose or hydroxypropyl methyl cellulose.

In an embodiment, the present subject matter provides a solid dispersion comprising venetoclax or a pharmaceutically acceptable salt thereof, co-povidone, hydroxypropyl cellulose and, optionally one or more pharmaceutically acceptable excipient, wherein the solid dispersion is devoid of any surfactant.

In an embodiment, the subject matter provides a solid dispersion comprising venetoclax or a pharmaceutically acceptable salt thereof, a water soluble polymer, and is free of a surfactant.

In an embodiment, the subject matter provides a solid dispersion comprising a water soluble polymer, a surfactant and is free of venetoclax or a pharmaceutically acceptable salt thereof.

In an embodiment, the subject matter provides a pharmaceutical composition comprising a mixture of at least two solid dispersions and optionally one or more pharmaceutically acceptable excipients, the first solid dispersion comprising venetoclax or a pharmaceutically acceptable salt thereof and a polymer, and the second solid dispersion comprising a polymer, a surfactant and is free of venetoclax or a pharmaceutically acceptable salt thereof. In a preferred embodiment, the first solid dispersion is free of a surfactant. In a further embodiment, the first and second solid dispersions comprise the same polymer. In a further preferred embodiment, the polymer is a water soluble polymer.

In an embodiment, the subject matter provides a pharmaceutical composition comprising a solid dispersion and optionally one or more pharmaceutically acceptable excipients, the solid dispersion comprising venetoclax or a pharmaceutically acceptable salt thereof, at least two polymers, and is free of a surfactant. In a preferred embodiment, the polymers are different. In a further preferred embodiment, the polymers are water soluble polymers.

### BRIEF DESCRIPTION OF THE DRAWING

FIG. 1 is an illustrative image of extrudates obtained by the method of Example 1A.

FIG. 2 is an illustrative image of extrudates obtained by the method of Example 1B.

FIG. 3 is an illustrative image of extrudates of Solid Dispersion I obtained by the method of Example 4.

FIG. 4 is an illustrative image of extrudates of Solid Dispersion II obtained by the method of Example 4.

FIG. 5 is an illustrative X-ray powder diffraction pattern of the venetoclax composition prepared in Example 4, the amorphous venetoclax prepared in Example 5, and a placebo composition similar to Example 4 without venetoclax.

### DETAILED DESCRIPTION OF THE SUBJECT MATTER

While various aspects and features of certain embodiments have been summarized above, the following detailed description illustrates a few exemplary embodiments in further detail to enable one of skill in the art to practice such embodiments. The described examples are provided for illustrative purposes and are not scope limiting.

In the following description, for the purposes of explanation, numerous specific details are set forth in order to provide a thorough understanding of the described embodiments. It will be apparent to one skilled in the art, however, that other aspects of the embodiments provided herein may be practiced without some of these specific details.

A pharmaceutical composition comprising venetoclax or a pharmaceutically acceptable salt thereof is provided. Further provided a process for preparing such composition.

The terms “composition”, “pharmaceutical composition” or “formulation” are synonymously used in the present specification and may be in the form of, e.g., tablet, capsule or granules.

The term “tablet” refers to tablet without a coating or tablet with one or more coatings. Furthermore, the term “tablet” comprises tablets having one, two, three or even more layers and press-coated tablets, wherein each of the before mentioned types of tablets may be without or with one or more coatings.

Unless otherwise indicated, all numbers used herein to express quantities, dimensions, and so forth used should be understood as being modified in all instances by the term “about.” As used in this specification, the singular forms “a”, “an”, and “the” include plural references unless the context clearly dictates otherwise. Thus, for example, a reference to “a process” includes one or more process, and/or steps of the type described herein and/or which will become apparent to those persons skilled in the art upon reading this disclosure and so forth

For the purpose of the present subject matter, active pharmaceutical ingredient (API) venetoclax is a base or a pharmaceutically acceptable salt or hydrate thereof and is in crystalline or amorphous forms thereof. However, the amorphous form of venetoclax base is preferred in a solid dispersion.

The terms pharmaceutically acceptable excipients, pharmaceutically compatible excipients, and excipients are used interchangeably in this disclosure. They refer to non-API substances such as diluents or fillers, disintegrating agents, binders, surfactants, lubricants and glidants used in formulating pharmaceutical products. They are generally safe for administering to humans according to established governmental standards, including those promulgated by the United States Food and Drug Administration.

The term “stability” refers to physical and/or chemical stability.

Physical stability of solid dispersion and/or composition comprising such solid dispersion refers to presence of amorphous form of venetoclax. Said solid dispersion and/or composition is considered to be stable if venetoclax is present in an amorphous form. Said solid dispersion and/or composition does not exhibit significant crystallinity.

In an embodiment, a solid dispersion of venetoclax or a pharmaceutically acceptable salt thereof and/or composition comprising such solid dispersion retains at least 90% w/w, at least 95% w/w, at least 99% w/w or about 100% w/w of venetoclax or a pharmaceutically acceptable salt thereof in amorphous form. Said solid dispersion and/or composition retains about 90% w/w, about 91% w/w, about 92% w/w, about 93% w/w, about 94% w/w, about 95% w/w, about 96% w/w, about 97% w/w, about 98% w/w, about 99% w/w or about 100% w/w of venetoclax or a pharmaceutically acceptable salt thereof in amorphous form.

In an embodiment, a solid dispersion of venetoclax or a pharmaceutically acceptable salt thereof and/or composition comprising such solid dispersion exhibit not more than 10% w/w of crystallinity, not more than 9% w/w of crystallinity, not more than 8% w/w of crystallinity, not more than 7% w/w of crystallinity, not more than 6% w/w of crystallinity, not more than 5% w/w of crystallinity, not more than 4% w/w of crystallinity, not more than 3% w/w of crystallinity, not more than 2% w/w of crystallinity, or not more than 1% w/w of crystallinity.

In an embodiment, a solid dispersion of venetoclax or a pharmaceutically acceptable salt thereof and/or composition comprising such solid dispersion lacks crystallinity at initial time point.

In an embodiment, a solid dispersion of venetoclax or a pharmaceutically acceptable salt and/or composition comprising such solid dispersion lacks crystallinity at least for 6 months when stored at 40° C./75% RH.

The term “chemical stability” relates to no or an acceptable level of drug-related impurities (also known as related substances) in terms of total impurity, maximum individual unknown impurity and single maximum individual impurity. Moreover, a pharmaceutical composition is defined to have chemical stability when active agents therein exhibit good stability as determined by a standard potency test such as assay and the like. A composition of the subject matter retains at least 90% w/w of the initial potency for at least 6 months when stored at 40° C./75% RH.

The term “impurity” relates to any compound having a retention time that differs from that of venetoclax or a pharmaceutically acceptable salt thereof by at least the detection limit of the chromatography apparatus used to determine the retention time. The different retention time may be measured, for example, by HPLC.

An aspect of the present subject matter is to provide a solid dispersion comprising venetoclax or a pharmaceutically acceptable salt thereof and one or more polymers.

In an embodiment, the weight percentage of venetoclax or a pharmaceutically acceptable salt thereof is about 1% w/w to about 20% w/w based on the total weight of the solid dispersion. Venetoclax or a pharmaceutically acceptable salt thereof may comprise about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, about 19% w/w or about 20% w/w based on the total weight of the solid dispersion. Venetoclax or a pharmaceutically acceptable salt thereof may be present in an amount of about 12% w/w to about 15% w/w based on the total weight of the solid dispersion.

In an embodiment, the solid dispersion disclosed herein is prepared using amorphous form of venetoclax or a pharmaceutically acceptable salt thereof.

A suitable polymer as per the present subject matter is a hydrophilic water soluble polymer, water insoluble polymer or enteric coating polymer and combinations thereof.

Examples of hydrophilic water soluble polymers for use in the solid dispersion include cellulose derivatives such as hydroxypropyl cellulose (e.g. marketed as Klucel), hydroxyethyl cellulose (e.g. marked as Natrosol), hydroxypropyl methyl cellulose (e.g. marketed as Methocel), methyl cellulose; vinyl based polymers such as Polyvinylpyrrolidone (e.g. marketed as Kollidon 30), copovidone (co-povidone) (e.g. marketed as Kollidon VA64); oligo and polysaccharides such as carrageenans, galactomannans and xanthan gum; cyclodextrins; polyethylene glycols; polyethylene oxides; copolymers and graft copolymers of polyethylene glycols or polyethylene oxides such as graft copolymer of polyethylene glycol, polyvinyl caprolactam and polyvinyl acetate (e.g., Soluplus®), and mixtures thereof.

Examples of water insoluble polymers for use in the solid dispersion include oils, beeswax, carnauba wax, microcrystalline wax, fatty alcohols, cetostearyl alcohol, stearyl alcohol, cetyl alcohol, myristyl alcohol, fatty acid esters, glyceryl monostearate, glycerol distearate, glycerol monooleate, acetylated monoglycerides, tristearin, tripalmitin, cetyl esters wax, glyceryl palmitostearate, glyceryl behenate, celluloses, ethylcellulose, low substituted hydroxypropyl cellulose (L-HPC), cellulose acylate, cellulose diacylate, cellulose triacylate, cellulose acetate, cellulose diacetate, cellulose triacetate, mono cellulose alkanylates, di cellulose alkanylates, tri cellulose alkanylates, mono cellulose arylates, di cellulose arylates, tricellulose arylates, mono cellulose alkenylates, di cellulose alkenylates, tri cellulose alkenylates, carboxymethylcellulose sodium (carmellose sodium), croscarmellose sodium, acacia, tragacanth, propylene glycol alginate, agar powder, gelatin, starch, partly pregelatinized starch, oil, sodium starch glycolate, phospholipid (lecithin), glucomannans, polymethacrylic acid based polymers, zein, aliphatic polyesters, Poly (D, L-lactide) (PLA), Poly (D,L-lactide, co-glycolide acid (PLGA), and mixtures thereof.

Examples of enteric polymers for use in the solid dispersion include cellulose acetate phthalate, cellulose acetate trimellitate, cellulose acetate succinate, methylcellulose phthalate, hydroxymethylcellulose ethyl phthalate, hydroxypropylmethylcellulose phthalate, hydroxypropylmethylcellulose acetate succinate, hydroxypropylmethyl acetate maleate, hydroxypropylmethyl trimellitate, carboxymethylethylcellulose, polyvinyl butyrate phthalate, polyvinyl alcohol acetate phthalate, methacrylic acid/ethyl acrylate copolymer, and methacrylic acid/methyl methacrylate copolymer, and combinations thereof.

In an embodiment, the weight percentage of polymer is about 35% w/w to about 90% w/w based on the total weight of the solid dispersion. Polymer may comprise about 40-85% w/w, about 40-80% w/w, about 50-85% w/w, about 50-80% w/w, about 55-85% w/w, about 55-80% w/w, about 60-85% w/w or about 60-80% w/w based on the total weight of the solid dispersion.

In an embodiment, the weight percentage of polymer in solid dispersion comprising of venetoclax or a pharmaceutically acceptable salt thereof is about 35% w/w to about 90% w/w based on the total weight of the solid dispersion. Polymer in solid dispersion comprising of venetoclax may contain about 40-85% w/w, about 40-80% w/w, about 50-85% w/w, about 50-80% w/w, about 55-85% w/w, about 55-80% w/w, about 60-85% w/w or about 60-80% w/w based on the total weight of the solid dispersion.

In an embodiment, the weight percentage of polymer in solid dispersion free of venetoclax or a pharmaceutically acceptable salt thereof is about 40% w/w to about 60% w/w based on the total weight of the solid dispersion. Polymer may be present in an amount of 50-55% w/w based on the total weight of the solid dispersion.

In an embodiment, the ratio of venetoclax or a pharmaceutically acceptable salt thereof to polymer in the solid dispersion is from about 1:1 to about 1:15. The ratio of venetoclax or a pharmaceutically acceptable salt thereof to polymer is about 1:1, about 1:2, about 1:3, about 1:4, about 1:5, about 1:6, about 1:7, about 1:8, about 1:9, about 1:10, about 1:11, about 1:12, about 1:13, about 1:14 or about 1:15.

In an embodiment, the solid dispersion comprising venetoclax or a pharmaceutically acceptable salt thereof and a polymer is devoid of a surfactant.

An aspect of the present subject matter is to provide a solid dispersion comprising venetoclax or a pharmaceutically acceptable salt thereof and at least two polymers.

In an embodiment, a solid dispersion comprises venetoclax or a pharmaceutically acceptable salt thereof, a first polymer, a second polymer, and optionally one or more pharmaceutically acceptable excipients.

In an embodiment, a solid dispersion comprises venetoclax or a pharmaceutically acceptable salt thereof, a first polymer, a second polymer, and optionally one or more pharmaceutically acceptable excipients, wherein the solid dispersion is devoid of any surfactant.

In an embodiment, the first polymer and the second polymer are not the same.

In an embodiment, the solid dispersion comprising two different polymers is free of surfactant.

In an embodiment, first and second polymers may be selected from hydrophilic water soluble polymers, enteric coating polymers and water insoluble polymers.

In an embodiment, the first polymer is a hydrophilic water soluble polymer and the second polymer is an enteric coating polymer.

In an embodiment, the first polymer is an enteric coating polymer and the second polymer is a hydrophilic water soluble polymer.

In an embodiment, both first polymer and second polymers are hydrophilic water soluble polymers.

In an embodiment, the second water soluble polymer is a hydrophilic water soluble polymer other than first water soluble polymer.

In an embodiment, the first polymer is a vinyl based polymer and second polymer is a cellulose derivative.

In an embodiment, the first polymer is co-povidone or povidone and second polymer is hydroxypropyl cellulose or hydroxypropyl methyl cellulose.

In an embodiment, the first polymer is co-povidone and second polymer is co-povidone.

In an embodiment, ratio of venetoclax or a pharmaceutically acceptable salt thereof to the first polymer in the solid dispersion is from about 1:1 to about 1:15. The ratio of venetoclax or a pharmaceutically acceptable salt thereof to the first polymer is about 1:1, about 1:2, about 1:3, about 1:4, about 1:5, about 1:6, about 1:7, about 1:8, about 1:9, about 1:10, about 1:11, about 1:12, about 1:13, about 1:14 or about 1:15.

In an embodiment, the ratio of venetoclax or a pharmaceutically acceptable salt thereof to the second polymer in the solid dispersion is from about 1:0.05 to about 1:10. The ratio of venetoclax or a pharmaceutically acceptable salt thereof to the second polymer is about 1:0.1 to about 1:5. The ratio of venetoclax or a pharmaceutically acceptable salt thereof to the second polymer is about 1:0.1, about 1:0.2, about 1:0.3, about 1:0.4, about 1:0.5, about 1:0.6, about 1:0.7, about 1:0.8, about 1:0.9, about 1:1, about 1:2, about 1:3, about 1:4, about 1:5.

In an embodiment, a solid dispersion of venetoclax or a pharmaceutically acceptable salt thereof comprises co-povidone as the first polymer.

In an embodiment, a solid dispersion of venetoclax or a pharmaceutically acceptable salt thereof comprises povidone as the first polymer.

In an embodiment, a solid dispersion of venetoclax or a pharmaceutically acceptable salt thereof comprises hydroxypropyl cellulose as the second polymer.

In an embodiment, a solid dispersion of venetoclax or a pharmaceutically acceptable salt thereof comprises hydroxypropyl methylcellulose as the second polymer.

In an embodiment, the present subject matter provides is a solid dispersion comprising venetoclax or a pharmaceutically acceptable salt thereof, co-povidone, hydroxypropyl cellulose and, optionally one or more pharmaceutically acceptable excipients, wherein the solid dispersion is devoid of any surfactant.

In an embodiment, a solid dispersion of venetoclax or a pharmaceutically acceptable salt thereof comprises co-povidone, hydroxypropyl cellulose and a glidant.

In an embodiment, a solid dispersion of venetoclax or a pharmaceutically acceptable salt thereof comprises povidone, hydroxypropyl cellulose and a glidant.

In an embodiment, a solid dispersion of venetoclax or a pharmaceutically acceptable salt thereof comprises co-povidone, hydroxypropyl methylcellulose and a glidant.

In an embodiment, a solid dispersion of venetoclax or a pharmaceutically acceptable salt thereof comprises povidone, hydroxypropyl methylcellulose and a glidant.

In an embodiment, ratio of second polymer to first polymer in the solid dispersion is from about 1:1 to about 1:100. The ratio of second polymer to first polymer in the solid dispersion is about 1:1, about 1:5, about 1:10, about 1:20, about 1:30, about 1:40, about 1:50, about 1:60, about 1:70, about 1:80, about 1:90 or about 1:100. The ratio of second polymer to first polymer in the solid dispersion is from about 1:1 to about 1:10. The ratio of second polymer to first polymer in the solid dispersion is about 1:1, about 1:2, about 1:3, about 1:4, about 1:5, about 1:6, about 1:7, about 1:8, about 1:9 or about 1:10.

Glidants aid in the processing of powder materials. Suitable glidants include, but not limited to silicon dioxide particularly colloidal silicon dioxide (e.g. Aerosil®, Cab-O-Sil®), calcium silicate, magnesium silicate, magnesium trisilicate, talc, starch, mixtures thereof or the like.

In an embodiment, a solid dispersion comprises venetoclax or a pharmaceutically acceptable salt thereof, co-povidone, hydroxypropyl cellulose and colloidal silicon dioxide wherein the solid dispersion is devoid of surfactant.

In an embodiment, a solid dispersion comprises about 1-10% w/w of glidant. The glidant is present in an amount of about 1% w/w, about 2% w/w, about 3% w/w, about 4% w/w, about 6% w/w, about 7% w/w, about 8% w/w, about 9% w/w or about 10% w/w based on the weight of solid dispersion. The glidants may be present in an amount of about 2% w/w based on the weight of solid dispersion.

In an embodiment, a solid dispersion comprising venetoclax comprises about 0.5-10% w/w of glidant. The glidant is present in an amount of about 0.5% w/w, 0.7% w/w, 1% w/w, about 2% w/w, about 3% w/w, about 4% w/w, about 6% w/w, about 7% w/w, about 8% w/w, about 9% w/w or about 10% w/w based on the weight of solid dispersion. The glidants may be present in an amount of about 2% w/w based on the weight of solid dispersion.

In an embodiment, a solid dispersion free of venetoclax comprises about 15-25% w/w of glidant based on the weight of solid dispersion.

In an embodiment, the solid dispersion disclosed herein may comprise a surfactant. Suitable surfactants useful in the present subject matter include anionic or non-ionic surfactants or mixtures.

Suitable anionic surfactant include alkyl sulfates (e.g., sodium lauryl sulfate), alkylcarboxylates, alkylbenzole sulfates, secondary alkane sulfonates, and mixtures thereof.

Suitable non-ionic surfactants includes polyoxyethylene castor oil derivatives such as PEG-35 castor oil, PEG-40 hydrogenated castor and PEG-60 hydrogenated castor oil; fatty acid monoesters of sorbitan, for example sorbitan monooleate (e.g., Span™ 80), sorbitan monostearate (e.g., Span™ 60), sorbitan monopalmitate (e.g., Span™ 40) and sorbitan monolaurate (e.g., Span™ 20); other fatty acid esters of sorbitan, for example, sorbitan tristearate and sorbitan trioleate; fatty acid monoesters of polyoxyethylene sorbitan (polysorbates) such as PEG-20 sorbitan monooleate (polysorbate 80, e.g., Tween™ 80) PEG-20 sorbitan monostearate (polysorbate 60, e.g., Tween™ 60), PEG-20 sorbitan monopalmitate (polysorbate 40, e.g., Tween™ 40), or PEG-20 sorbitan monolaurate (polysorbate 20, e.g., Tween™ 20); other fatty acid esters of polyoxyethylene sorbitan, for example, polyoxyethylene (20) sorbitan tristearate (Tween 65), polyoxyethylene (20) sorbitan trioleate (Tween 85); fatty acid ester of polyalkylene glycols such as, for example, PEG 660 hydroxy-stearic acid (polyglycol ester of 12-hydroxystearic acid (70 mol %) with 30 mol % ethylene glycol); polyalkoxylated ethers of fatty alcohols such as, for example, PEG (2) stearyl ether (Brij 72), macrogol 6 cetylstearyl ether or macrogol 25 cetylstearyl ether; tocopherol compounds such as α-tocopheryl polyethylene glycol succinate (Vitamin E TPGS); or mixtures thereof.

In an embodiment, surfactant is present as a part of solid dispersion comprising venetoclax or a pharmaceutically acceptable salt thereof, one or more polymers.

In an embodiment, solid dispersion of surfactant and one or more polymers is prepared in addition to solid dispersion comprising venetoclax or a pharmaceutically acceptable salt thereof and one or more polymers. That is, the one solid dispersion comprises one or more polymers and at least one surfactant but no venetoclax or a pharmaceutically acceptable salt thereof, while the solid dispersion comprises venetoclax or a pharmaceutically acceptable salt thereof and one or more polymers with or without surfactant. In a preferred embodiment, the second solid dispersion is free of surfactant.

In an embodiment, the solid dispersion comprises venetoclax or a pharmaceutically acceptable salt thereof, one or more polymers, surfactant and a pharmaceutically acceptable excipient.

In an embodiment, the solid dispersion disclosed herein is devoid of glidant.

An aspect of the present subject matter is to provide a solid dispersion comprising venetoclax or a pharmaceutically acceptable salt thereof with improved manufacturability.

In certain embodiments, solid dispersion is prepared by suitable technique such as a melting process including co-melting, an extrusion technique such as hot melt extrusion or a solvent removal process. The solvent removal process may include without limitation solvent evaporation, rotary evaporation and spray-drying.

In a preferred embodiment, the solid dispersion is prepared by hot melt extrusion of venetoclax or a pharmaceutically acceptable salt thereof with one or more polymers. In a further preferred embodiment, the input venetoclax or a pharmaceutically acceptable salt thereof is amorphous, or substantially so.

With respect to hot melt extrusion, the melting and mixing takes place in extruders or kneaders. Suitable extruders include single-screw extruders and multi-screw extruders, for example twin-screw extruders, which can be co-rotating or counter-rotating and, optionally, equipped with kneading disks or other screw elements for mixing or dispersing the components of the melt.

The elevated temperature attained during this part of the process can suitably be between about 70° C. to about 200° C., such as for example about 70° C. to about 180° C., about 70° C. to about 160° C., about 80° C. to about 180° C., about 80° C. to about 160° C., about 90° C. to about 180° C., about 90° C. to about 160° C., about 100° C. to about 180° C., and 100° C. to about 160° C.

In an aspect, a process of preparing a solid dispersion comprising venetoclax or a pharmaceutically acceptable salt thereof comprises the steps of:
a) Mixing and co-sifting venetoclax or a pharmaceutically acceptable salt thereof, a first polymer and second polymer,
b) Blending the sifted material of step (a), and
c) Loading the blend of step (b) into twin screw hot melt extruder and making the extrudes with suitable parameters.

In a preferred embodiment, the process comprises the steps of:
a) Mixing and co-sifting venetoclax or a pharmaceutically acceptable salt thereof, a first hydrophilic water soluble polymer, a second different hydrophilic water soluble polymer, and a glidant, without surfactant
b) Blending the sifted material of step (a), and
c) Loading the blend of step (b) into a twin screw hot melt extruder Pharma 11 by Thermo Scientific and making the extrudes with suitable parameters including a zone temperature ranging from 700 C to 1600 C.

In an aspect, a process of preparing a solid dispersion comprising venetoclax or a pharmaceutically acceptable salt thereof comprises the steps of:
a) Sifting a polymer,
b) Co-sifting venetoclax or a pharmaceutically acceptable salt thereof with material of step a),
c) Re-sifting material of step b) and blending, and
d) Loading the blend of step (c) into a twin screw hot melt extruder and making the extrudes with suitable parameters.

In a preferred embodiment, the process comprises the steps of:
a) Co-sifting a hydrophilic water soluble polymer and a glidant,
b) Co-sifting venetoclax or a pharmaceutically acceptable salt thereof with material of step a),
c) Re-sifting material of step b) and blending, and
d) Loading the blend of step (b) into a twin screw hot melt extruder Pharma 11 by Thermo Scientific and making the extrudes with suitable parameters including a zone temperature ranging from 800 C to 1600 C.

In an aspect, a process of preparing a solid dispersion free of venetoclax comprises the steps of:
a) Sifting a polymer,
b) Mixing a surfactant and a glidant,
c) Co-sifting of mixture (b) with polymer of step (a) and blending, and
d) Loading the blend of step (c) into twin screw hot melt extruder and making the extrudes with suitable parameters.

In a preferred embodiment, the process comprises the steps of:
a) Sifting a hydrophilic water soluble polymer and a glidant separately,
b) Adsorbing a surfactant onto the glidant of step a) in a rapid mixer granulator,
c) Co-sifting granules of step b) with the hydrophilic water soluble polymer of step a) and blending, and
d) Loading the blend of step (c) into a twin screw hot melt extruder of Pharma 11 by Thermo Scientific and making the extrudes with suitable parameters including a zone temperature ranging from 800 C to 1300 C.

Without bound by any theory, it is hypothesized that less residence time may lead to less impurity levels (related substances). Accordingly, usage of two water soluble polymers provided better feed rate and higher screw speed because of less torque, which decreased the residence time of the mixture inside the machine, thus leading to production of less related substances.

In an embodiment, the present subject matter results in lesser time for processing and thus enables faster manufacturability and scalability of the extrudates.

Manufacturability of the extrudates can be evaluated using generated torque during manufacturing, which is indicative of the ease with which the input material is processed. Manufacturability can also be evaluated by appearance of extrudates, i.e. clarity, which is indicative of homogeneity of solid dispersion.

Torque is the resistance provided by the material to the screw in the hot melt extruder during melting process. Generally, the equipment will stop when torque reaches 100%. Moreover, torque can vary with different compositions. Therefore, at predetermined zone temperature, less torque value even at higher screw speed indicates that the process is smooth. Less torque value implies the selection of higher feed rate and faster screw speed. In other words, manufacturability is also dependent on the choice of composition.

The clarity of extrudates is indicative of complete molecular dispersion of venetoclax in the polymer matrix. The better clarity of extrudes directly affects the nature of the molecular dispersion, which in turn increases solubility of the API and improves bioavailability.

Moreover, the faster feed rate reduces the process time, resulting in higher production per unit time, which in turn increases scalability of the process.

In one embodiment, it was found that addition of a different water soluble second polymer in solid dispersion comprising a first water soluble polymer may result in one or more of following with regard to a venetoclax composition:
1) Improved dissolution,
2) Improved chemical stability (for example, reduced relative substances),
3) Improved stability of amorphous venetoclax (for example, less crystallinity),
4) Improved manufacturability,
5) Bio-equivalency with VENCLEXTA®, which contains a solid dispersion component containing venetoclax, a water soluble polymer and a surfactant.

It was found that addition of a second different hydrophilic water soluble polymer to a first hydrophilic water soluble polymer in solid dispersion may result in one or more of following with regard to a venetoclax composition:
1) Less torque value at higher screw speed indicating smooth processing,
2) Improved clarity of extrudates indicating complete molecular dispersion of venetoclax in the polymer matrix,
3) Improved dissolution,
4) Improved stability indicating less impurity generation.

In another embodiment, the presence of two solid dispersions in the venetoclax composition, i.e., a first solid dispersion comprising venetoclax, a water soluble polymer and free of surfactant, and a second solid dispersion comprising a water soluble polymer, a surfactant and free of venetoclax, may provide improved dissolution/bioavailability compared to a composition comprising one solid dispersion. It may also resolve the incompatibility of venetoclax with surfactant/s as both are part of separate solid dispersions. Additionally, such a system is likely to avoid the need of using two separate polymers.

An aspect of the present subject matter is to provide a pharmaceutical composition comprising a solid dispersion comprising venetoclax or a pharmaceutically acceptable salt thereof and one or more polymers.

An aspect of the present subject matter is to provide a pharmaceutical composition comprising a solid dispersion comprising venetoclax or a pharmaceutically acceptable salt thereof and at least two polymers.

An aspect of the present subject matter is to provide a pharmaceutical composition comprising a solid dispersion comprising venetoclax or a pharmaceutically acceptable salt thereof and at least two polymers, wherein the pharmaceutical composition is bioequivalent to VENCLEXTA®.

In an embodiment, the composition comprises solid dispersion of venetoclax and at least one pharmaceutically acceptable excipient selected from polymer, diluents, disintegrating agents, binders, surfactants, lubricants and glidants.

In an embodiment, a composition comprises solid dispersion of venetoclax or pharmaceutically acceptable salt thereof and a polymer.

In an embodiment, the weight percentage of venetoclax or a pharmaceutically acceptable salt thereof is about 1% w/w to about 15% w/w based on the total weight of the composition. Venetoclax or a pharmaceutically acceptable salt thereof may comprise about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w or about 15% w/w based on the total weight of the composition. Venetoclax or a pharmaceutically acceptable salt thereof may be present in an amount of about 10% w/w based on the total weight of the composition. Venetoclax or a pharmaceutically acceptable salt thereof is preferably present in the composition in a mass amount of 10 mg, 50 mg or 100 mg.

Suitable polymers as per the present subject matter are hydrophilic water soluble polymers, water insoluble polymers, enteric coating polymers, and combinations thereof.

In an embodiment, the weight percentage of the polymer is about 35% w/w to about 90% w/w based on the total weight of the composition. The polymer comprises about 40-85% w/w, about 40-80% w/w, about 50-85% w/w, about 50-80% w/w, about 55-85% w/w, about 55-80% w/w, about 60-85% w/w or about 60-80% w/w, about 65-85% w/w or about 65-80% w/w based on the total weight of the composition. The polymer comprises about 65% w/w, about 70% w/w, about 75% w/w, about 80% w/w or about 85% w/w based on the total weight of the composition. The polymer may be present in an amount of about 70% w/w based on the total weight of the composition.

In an embodiment, a composition comprises a solid dispersion of venetoclax and a diluent.

Examples of suitable diluents (also referred to as fillers) as per the present subject matter are lactose, in particular lactose monohydrate, cellulose and derivatives, such as powdered cellulose, microcrystalline or silicified microcrystalline cellulose, cellulose acetate, starches and derivatives such as pregelatinized starch, corn starch, wheat starch, rice starch, potato starch, sterilizable maize, sodium chloride, calcium carbonate, calcium phosphate, particularly dibasic calcium phosphate, calcium sulphate, dicalcium or tricalcium phosphate, magnesium carbonate, magnesium oxide, sugars and derivatives such as confectioner's sugar, fructose, sucrose, dextrates, dextrin, D-sorbitol sulfobutylether O-cyclodextrin, dextrose, polydextrose, trehalose, maltose, maltitol, mannitol, maltodextrin, sorbitol, inulin, xylitol, erythritol, isomalt, kaolin, lactitol, and mixtures thereof.

In an embodiment, a pharmaceutical composition comprises solid dispersion of venetoclax and microcrystalline cellulose.

In an embodiment, a pharmaceutical composition comprises about 5-50% w/w of diluents based on total weight of the composition. Diluents comprise an amount of about 5-40% w/w, about 5-30% w/w, about 5-20% w/w, or about 5-10% w/w based on total weight of the composition. Diluents comprise an amount of about 5% w/w, about 6% w/w, about 7% w/w, about 8% w/w, about 9% w/w or about 10% w/w based on total weight of the composition.

Diluents may be present in an amount of about 7% w/w based on total weight of the composition.

In an embodiment, a pharmaceutical composition comprising a first solid dispersion with venetoclax and a second solid dispersion without venetoclax, may comprise about 2-50% w/w of diluents based on total weight of the composition. Diluents comprise an amount of about 2-40% w/w, about 2-30% w/w, about 2-20% w/w, or about 2-10% w/w based on total weight of the composition. Diluents comprise an amount of about 2% w/w, 3% w/w, 4% w/w, 5% w/w, about 6% w/w, about 7% w/w, about 8% w/w, about 9% w/w or about 10% w/w based on total weight of the composition. Diluents may be present in an amount of about 7% w/w based on total weight of the composition.

In an embodiment, a composition comprises a solid dispersion of venetoclax and a surfactant.

In an embodiment, a composition comprises a solid dispersion of venetoclax and a lubricant.

In an embodiment, a composition comprises a solid dispersion of venetoclax and a glidant.

In an embodiment, a composition comprises a solid dispersion of venetoclax, a diluent and a polymer.

In an embodiment, a composition comprises a solid dispersion of venetoclax, a diluent and a surfactant.

In an embodiment, a composition comprises a solid dispersion of venetoclax, a diluent and a lubricant.

In an embodiment, a composition comprises a solid dispersion of venetoclax, a diluent and a glidant.

In an embodiment, a composition comprises a solid dispersion of venetoclax, a polymer and a surfactant.

In an embodiment, a composition comprises a solid dispersion of venetoclax, a polymer and a lubricant.

In an embodiment, a composition comprises a solid dispersion of venetoclax, a polymer and a glidant.

In an embodiment, a composition comprises a solid dispersion of venetoclax, a surfactant and a lubricant.

In an embodiment, a composition comprises a solid dispersion of venetoclax, a surfactant and a glidant.

In an embodiment, a composition comprises a solid dispersion of venetoclax, a glidant and a lubricant.

In an embodiment, a composition comprises a solid dispersion of venetoclax, a diluent, a polymer and a surfactant.

In an embodiment, a composition comprises a solid dispersion of venetoclax, a diluent, a polymer and a lubricant.

In an embodiment, a composition comprises a solid dispersion of venetoclax, a diluent, a polymer and a glidant.

In an embodiment, a composition comprises a solid dispersion of venetoclax, a diluent, a surfactant and a lubricant.

In an embodiment, a composition comprises a solid dispersion of venetoclax, a diluent, a surfactant and a glidant.

In an embodiment, a composition comprises a solid dispersion of venetoclax, a diluent, a glidant and a lubricant.

In an embodiment, a composition comprises a solid dispersion of venetoclax, a diluent, a polymer, a surfactant and a lubricant.

In an embodiment, a composition comprises a solid dispersion of venetoclax, a diluent, a polymer, a surfactant and a glidant.

In an embodiment, a composition comprises a solid dispersion of venetoclax, a diluent, a polymer, a glidant and a lubricant.

In an embodiment, a composition comprises a solid dispersion of venetoclax, a diluent, a surfactant, a glidant and a lubricant.

In an embodiment, a composition comprises a solid dispersion of venetoclax, a diluent, a polymer, a surfactant, a lubricant and a glidant.

In an aspect, a composition comprises a mixture of a solid dispersion comprising venetoclax and a solid dispersion free of venetoclax.

In an embodiment, the solid dispersion comprising venetoclax comprises venetoclax, a water soluble polymer and optionally, a glidant.

In an embodiment, the solid dispersion free of venetoclax comprises a water soluble polymer, a surfactant and optionally, a glidant.

In an embodiment, the solid dispersion comprising venetoclax is free of surfactant.

In an embodiment, the water soluble polymer present in the solid dispersion comprising venetoclax and the solid dispersion free of venetoclax is the same polymer.

In an embodiment, the water soluble polymer present in the solid dispersion comprising venetoclax and the solid dispersion free of venetoclax is copovidone.

In an embodiment, the solid dispersion free of venetoclax comprises polysorbate 80 as a surfactant.

Suitable surfactant useful in the present subject matter include anionic or non-ionic surfactants or mixtures. Suitable anionic surfactant include alkyl sulfates (e.g., sodium lauryl sulfate), alkylcarboxylates, alkylbenzole sulfates, secondary alkane sulfonates and mixtures thereof.

Suitable non-ionic surfactants includes polyoxyethylene castor oil derivatives such as PEG-35 castor oil, PEG-40 hydrogenated castor and PEG-60 hydrogenated castor oil; fatty acid

monoesters of sorbitan, for example sorbitan monooleate (e.g., Span™ 80), sorbitan monostearate (e.g., Span™ 60), sorbitan monopalmitate (e.g., Span™ 40) and sorbitan monolaurate (e.g., Span™ 20); other fatty acid esters of sorbitan, for example, sorbitan tristearate and sorbitan trioleate; fatty acid monoesters of polyoxyethylene sorbitan (polysorbates) such as PEG-20 sorbitan monooleate (polysorbate 80, e.g., Tween™ 80) PEG-20 sorbitan monostearate (polysorbate 60, e.g., Tween™ 60), PEG-20 sorbitan monopalmitate (polysorbate 40, e.g., Tween™ 40), or PEG-20 sorbitan monolaurate (polysorbate 20, e.g., Tween™ 20); other fatty acid esters of polyoxyethylene sorbitan, for example, polyoxyethylene (20) sorbitan tristearate (Tween 65), polyoxyethylene (20) sorbitan trioleate (Tween 85); fatty acid ester of polyalkylene glycols such as, for example, PEG 660 hydroxy-stearic acid (polyglycol ester of 12-hydroxystearic acid (70 mol %) with 30 mol % ethylene glycol); polyalkoxylated ethers of fatty alcohols such as, for example, PEG (2) stearyl ether (Brij 72), macrogol 6 cetylstearyl ether or macrogol 25 cetylstearyl ether; tocopherol compounds such as α-tocopheryl polyethylene glycol succinate (Vitamin E TPGS); or mixtures thereof.

As per the prior art, although higher surfactant level leads to faster drug release rate, greater surfactant level may affect the manufacturability of the extrudates in the preparation of solid dispersions.

In an embodiment, usage of second polymer in the solid dispersion compensated for the problem associated with usage of greater surfactant level, i.e., as per the present subject matter, manufacturability of the extrudates in the preparation of solid dispersions remains unaffected by the amount of surfactant used because of usage of the second polymer.

In an embodiment, a composition comprises about 1-10% w/w of surfactant based on total weight of the composition. Surfactant may be present in an amount of about 1% w/w, about 2% w/w, about 3% w/w, about 4% w/w, about 5% w/w, about 6% w/w, about 7% w/w, about 8% w/w, about 9% w/w or about 10% w/w of surfactant based on total weight of the composition. Surfactant may be present in an amount of about 5% w/w based on total weight of the composition.

In an embodiment, a composition comprises a first solid dispersion comprising venetoclax or pharmaceutically acceptable salt thereof and at least two polymers and a second solid dispersion comprising surfactant and at least one polymer.

In an embodiment, a composition comprises a first solid dispersion comprising venetoclax or pharmaceutically acceptable salt thereof, co-povidone and hydroxypropyl cellulose and a second solid dispersion comprising surfactant and co-povidone.

In an embodiment, a composition comprises a first solid dispersion comprising venetoclax or pharmaceutically acceptable salt thereof, co-povidone and hydroxypropyl cellulose and a second solid dispersion comprising surfactant, co-povidone and hydroxypropyl cellulose.

Suitable lubricants according to the subject matter are talc, sodium stearate, calcium stearate, zinc stearate, magnesium stearate, sodium stearyl fumarate, glyceryl monostearate, polyethylene glycols, hydrogenated castor oil, fatty acid, for example fumaric acid, and salts of fatty acids, in particular the calcium, magnesium, sodium or potassium salts thereof, for example calcium behenate, calcium stearate, sodium stearyl fumarate or magnesium stearate, and glycerides such as glyceryl behenate (Compritol® 888), Dynasan® 118 or Boeson® VP).

In an embodiment, a composition comprises 0.20-2.5% w/w of lubricant based on the total weight of the composition. Lubricant may be present in an amount of 0.2-2.0% w/w, 0.3-1.8% w/w, 0.4-1.5% w/w, or 0.5-1% w/w of based on the total weight of the composition. Lubricant may be present in an amount of 0.5% w/w based on the total weight of the composition.

In an embodiment, a composition comprises about 1-10% w/w of glidant based on the total weight of the composition. Glidant may be present in an amount of about 1% w/w, about 2% w/w, about 3% w/w, about 4% w/w, about 6% w/w, about 7% w/w, about 8% w/w, about 9% w/w or about 10% w/w based on the total weight of the composition. Glidant may be present in an amount of 5% w/w based on the total weight of the composition.

In an embodiment, a composition comprises solid dispersion of venetoclax and disintegrating agent and another pharmaceutically acceptable excipient.

Examples of disintegrating agent may be selected from sodium starch glycolate croscarmellose sodium, croscarmellose calcium, crospovidone, docusate sodium, low-substituted hydroxypropyl cellulose, magnesium aluminum silicate, microcrystalline cellulose, polacrilin potassium, and starch, particularly pregelatinized starch and corn starch.

In an embodiment, a pharmaceutical composition is in the form of tablet.

A dosage form according to this subject matter, such as a tablet, or film-coated tablet, may be prepared by methods well-known to the one skilled in the art like direct compression, wet granulation and dry granulation. Suitable methods of manufacturing a tablet include compression of the pharmaceutical composition in the form of a powder, i.e. direct compression, or wet granulation or dry granulation compression of the pharmaceutical composition in the form of granules, and if needed with additional excipients.

An aspect of the subject matter provides a process for preparing composition comprising the solid dispersion of venetoclax including hot melt extrusion technique.

In an embodiment, a process for preparing pharmaceutical composition comprising solid dispersion of venetoclax comprises the steps of:
a) Mixing and co-sifting venetoclax, a first polymer and a second polymer
b) Blending the sifted material of step (a) for 15 minutes,
c) Loading the blend of step (b) into a twin screw hot melt extruder and making the extrudes with suitable parameters,
d) Milling the extrudes of step (c) in a suitable mill and sifting through a suitable sieve,
e) Optionally, adsorbing any liquid excipient if present onto any of the extra-granular excipients and granulating the same,
f) Sifting the extra-granular excipients (along with granules of step (e) if present) through a suitable sieve,
g) Mixing materials of step (d) and step (f) in a blender for a suitable period of time,
h) Lubricating the powder blend of step (g),
i) Compressing the lubricated blend of step (g) using a designated tooling set, and
j) Film coating the tablets of step (h) using a desired coating material.

In an embodiment, presence of two hydrophilic water soluble polymer such as co-povidone and hydroxypropyl cellulose in solid dispersion improved manufacturability of the extrudates, and the dissolution profile and stability of compositions compared to the solid dispersions with a single hydrophilic water soluble polymer.

A tablet according to the subject matter may be film-coated. Typically a film coat represents 2-5% by weight of the total composition and comprises preferably a film-forming agent, a plasticizer, an anti-tacking agent and optionally one or more pigments.

Examples of film-forming agents include are polyvinylpyrrolidone, natural gums, starches, and cellulosic polymers. A cellulosic polymer may include a molecule comprising at least one cellulose polymer or derivative modified with small amounts of propylene glycol ether groups attached to the cellulose anhydroglucose chain affording binding properties that enhance the reinforcing film properties of film applications. Examples of cellulosic polymers include, but are not limited to, hydroxypropyl methyl cellulose (“HPMC), carboxymethyl cellulose (“CMC”) or salts thereof, hydroxypropyl cellulose (“HPC), methylcellulose (“MC), hydroxyethyl cellulose (“HEC), and the like. In addition, cellulosic polymers may be characterized as ionic or non-ionic. Ionic cellulosic polymers include, for example, sodium CMC. Non-ionic cellulosic polymers include, for example, HPMC, HPC, HEC, and MC.

In a preferred embodiment, a pharmaceutical composition comprises an intragranular portion comprising a) a first solid dispersion free of surfactant comprising venetoclax or a pharmaceutically acceptable salt thereof (preferably venetoclax), a single water soluble polymer (preferably copovidone) and a glidant (preferably colloidal silicon dioxide), and b) a second solid dispersion free of venetoclax or a pharmaceutically acceptable salt thereof comprising a single water soluble polymer (preferably copovidone), a surfactant (preferably polysorbate 80) and a glidant (preferably colloidal silicon dioxide). The composition preferably further comprises an extragranular portion comprising a filler (preferably microcrystalline cellulose) and a glidant (preferably colloidal silicon dioxide). The composition preferably contains a lubricant (preferably sodium stearyl fumarate). The composition is preferably in the form of a tablet (preferably film-coated). The composition ideally shows in vitro and in vivo bioequivalence to VENCLEXTA® as per the FDA's guidances.

In another preferred embodiment, a pharmaceutical composition comprises an intragranular portion comprising a solid dispersion free of surfactant comprising venetoclax or a pharmaceutically acceptable salt thereof (preferably venetoclax), two different water soluble polymers (preferably copovidone and HPC or HPMC) and a glidant (preferably colloidal silicon dioxide). The composition preferably further comprises an extragranular portion comprising a filler (preferably microcrystalline cellulose), a surfactant (preferably polysorbate 80) and a glidant (preferably colloidal silicon dioxide). The composition preferably contains a lubricant (preferably sodium stearyl fumarate). The composition is preferably in the form of a tablet (preferably film-coated). The composition ideally shows in vitro and in vivo bioequivalence to VENCLEXTA® as per the FDA's guidances.

In an embodiment, a composition comprising solid dispersion of venetoclax is suitable in treatment of a disease characterized by overexpression of one or more anti-apoptotic Bcl-2 family proteins, for example cancer or an immune or autoimmune disease. Dosage amounts and are regimens are well known in the art.

### EXAMPLES

### Example 1

TABLE 1

| | | |
|---|---|---|
| | Example 1A | Example 1B |
| Ingredients | % w/w | % w/w |

Solid Dispersion

| | | |
|---|---|---|
| Venetoclax | 10.121 | 10.121 |
| Co-povidone | 67.470 | 67.470 |
| Hydroxypropyl cellulose | 1.012 | — |
| Colloidal silicon dioxide | 1.913 | 1.913 |

Outside dispersion

| | | |
|---|---|---|
| Polysorbate 80 | 4.721 | 4.721 |
| Colloidal silicon dioxide | 3.147 | 3.147 |
| Microcrystalline cellulose | 7.233 | 8.245 |
| Sodium Stearyl fumarate | 0.536 | 0.536 |
| Core tablet weight | 96.154 | 96.154 |
| Film coating | 3.846 | 3.846 |
| Total tablet weight | 100.00 | 100.00 |

Manufacturing Process:
a) Venetoclax, co-povidone, hydroxypropyl cellulose (if present) and colloidal silicon dioxide were mixed and co-sifted,
b) sifted material of step (a) was blended for 15 minutes,
c) blend of step (b) was loaded into twin screw hot melt extruder and extrudes were made,
d) extrudes of step (c) were milled in a suitable mill and sifted through suitable sieve to obtain granules of desired size,
e) polysorbate 80 was adsorbed onto colloidal silicon dioxide in a rapid mixer granulator using suitable parameters and resulting granules were sifted through suitable sieve.
f) granules of step (e) and microcrystalline cellulose were sifted through suitable sieve,
g) materials of step (d) and step (f) were mixed in a blender for suitable period of time,
h) powder blend of step (g) was lubricated with sifted sodium stearyl fumarate,
i) lubricated blend of step (h) was compressed into tablets using designated tooling set, and
j) tablets of step (i) were film coated using desired coating material.

### Example 2

TABLE 2

| | |
|---|---|
| Ingredients | Quantity (% w/w) |

Solid Dispersion

| | |
|---|---|
| Venetoclax | 10.121 |
| Hydroxypropyl cellulose | 5.060 |
| Colloidal silicon dioxide | 1.913 |

Outside dispersion

| | |
|---|---|
| Polysorbate 80 | 5.900 |
| Colloidal silicon dioxide | 3.934 |
| Microcrystalline cellulose | 68.687 |
| Sodium Stearyl fumarate | 0.536 |
| Core tablet weight | 96.154 |
| Film coating | 3.846 |
| Total tablet weight | 100.00 |

Manufacturing Process:
a) Venetoclax, hydroxypropyl cellulose and colloidal silicon dioxide were mixed and co sifted,
b) sifted material of step (a) was blended for 15 minutes,
c) blend of step (b) was loaded into twin screw hot melt extruder and extrudes were made,
d) extrudes of step (c) were milled in a suitable mill and sifted through suitable sieve to obtain granules of desired size,
e) polysorbate 80 was adsorbed onto colloidal silicon dioxide in a rapid mixer granulator using suitable parameters and resulting granules were sifted through suitable sieve.
f) granules of step (e) and microcrystalline cellulose were sifted through suitable sieve,
g) materials of step (d) and step (f) were mixed in a blender for suitable period of time,
h) powder blend of step (g) was lubricated with sifted sodium stearyl fumarate,
i) lubricated blend of step (h) was compressed into tablets using designated tooling set, and
j) tablets of step (i) were film coated using desired coating material.

### Example 3

TABLE 3

| | |
|---|---|
| Ingredients | Quantity (% w/w) |

Solid Dispersion

| | |
|---|---|
| Venetoclax | 10.121 |
| Co-povidone | 67.470 |
| Hydroxypropyl methylcellulose | 1.012 |
| Colloidal silicon dioxide | 1.913 |

Outside dispersion

| | |
|---|---|
| Polysorbate 80 | 4.721 |
| Colloidal silicon dioxide | 3.147 |
| Microcrystalline cellulose | 7.233 |
| Sodium Stearyl fumarate | 0.536 |
| Core tablet weight | 96.154 |
| Film coating | 3.846 |
| Total tablet weight | 100.00 |

Manufacturing Process:
a) Venetoclax, co-povidone, hydroxypropyl methylcellulose and colloidal silicon dioxide were mixed and co-sifted,
b) sifted material of step (a) was blended for 15 minutes,
c) blend of step (b) was loaded into twin screw hot melt extruder and extrudes were made,
d) extrudes of step (c) were milled in a suitable mill and sifted through suitable sieve to obtain granules of desired size,
e) polysorbate 80 was adsorbed onto colloidal silicon dioxide in a rapid mixer granulator using suitable parameters and resulting granules were sifted through suitable sieve.
f) granules of step (e) and microcrystalline cellulose were sifted through suitable sieve,
g) materials of step (d) and step (f) were mixed in a blender for suitable period of time,
h) powder blend of step (g) was lubricated with sifted sodium stearyl fumarate,
i) lubricated blend of step (h) was compressed into tablets using designated tooling set, and
j) tablets of step (i) were film coated using desired coating material.

Parameters for Hot Melt Extrusion from Examples 1 to 3:

TABLE 4

| Parameter | Acceptance criteria |
|---|---|
| Description | Pale yellow to yellow colored clear extrudates |
| Die Size | 2 mm |
| Screw speed | 50-250 rpm |
| Zone temperature | 70-160° C. |

Manufacturability Studies

It was observed that processability and formulation characteristics were improved when an additional water soluble polymer (hydroxypropyl cellulose) is used inside solid dispersion compared to the solid dispersion without it. In particularly, the improvement is observed with respect to torque, clarity and dissolution studies as follows:

a. Torque:

TABLE 5

| | Value | |
|---|---|---|
| Attribute | Example 1A | Example 1B |
| Torque value | Low (<70%) at higher | High (>70%) at lower |
| | screw speed (250 rpm) | screw speed (150 rpm) |
| Throughput | High | Low |

Torque value was less than 70% in Example 1A at zone temperature range of 90° C. to 155° C. (even at screw speed of 250 rpm), indicating that the process can run smoothly at even higher screw speed greater than 250 rpm. Hence throughput was higher upon addition of hydroxypropyl cellulose to the copolymer inside solid dispersion. Without hydroxypropyl cellulose inside solid dispersion, higher torque was observed (>70%) at zone temperature range of 90° C. to 155° C. (even at screw speed of 150 rpm), indicating that the process was not smooth above 150 rpm and throughput was lower.

b. Clarity:

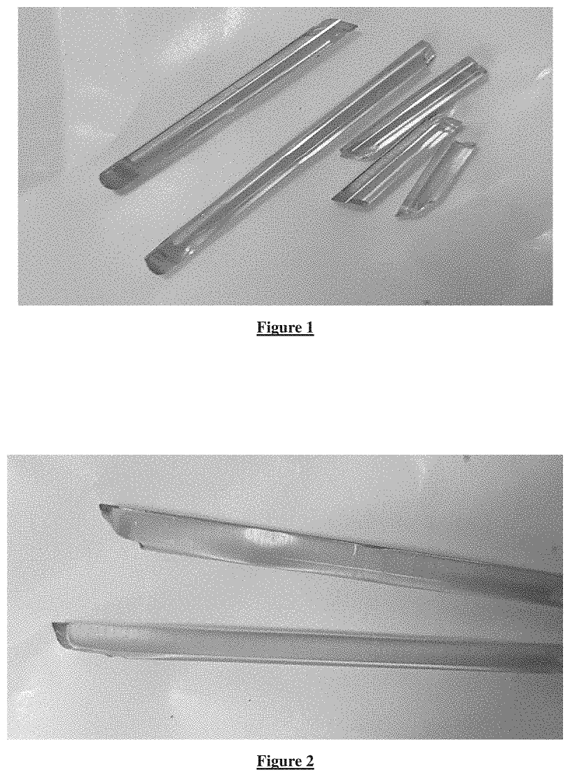

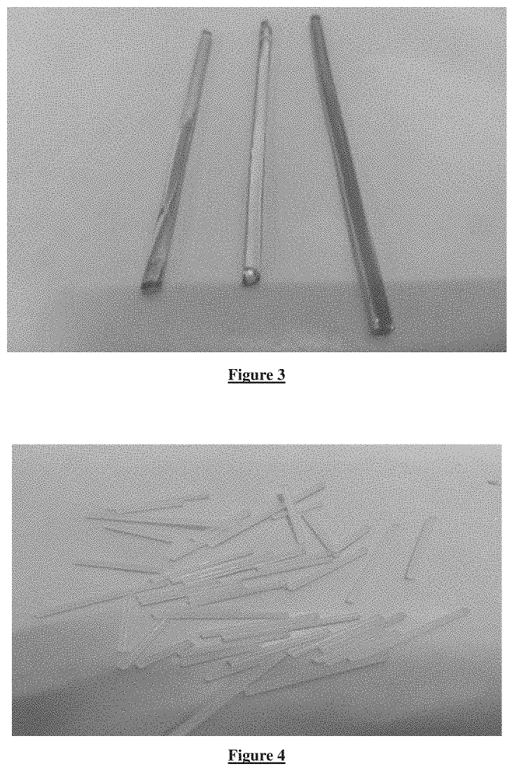

Extrudes of venetoclax made with hydroxypropyl cellulose in Example 1A had improved clarity in comparison to extrudes without hydroxypropyl cellulose in Example 1B, which indicated complete and homogenous molecular dispersion of venetoclax API in the polymer matrix. The difference can be observed in the accompanying extrudate images obtained from Examples 1A and 1B in FIGS. 1 and 2, respectively.

c. Dissolution Studies:

Tablets prepared as per Example 1 were subjected to dissolution in 900 mL of pH 6.8 phosphate buffer with 0.4% sodium dodecyl sulphate, USP Type II apparatus (paddle) at 75 rpm. Results are as provided below:

TABLE 6

| Time | % Drug released | |
|---|---|---|
| (in hours) | Example 1A | Example 1B |
| 1 | 7 | 6 |
| 2 | 20 | 17 |
| 4 | 48 | 39 |
| 6 | 71 | 63 |
| 8 | 86 | 81 |
| 10 | 94 | 93 |
| 12 | 97 | 100 |

As observed from the above Table 6, the release of venetoclax from the tablets at time points 4, 6 and 8 hours are distinguishably greater for Example 1A (tablets with hydroxypropyl cellulose inside solid dispersion) compared to example 1B (without hydroxypropyl cellulose inside solid dispersion).

Tablets prepared as per Example 1 were also subjected to dissolution in 250 mL of pH 4.5 acetate buffer with 0.4% sodium dodecyl sulphate, USP Type III apparatus (reciprocating cylinder) at 20 dpm with bottom screen of 40 mesh stainless steel. Results are as provided below:

TABLE 7

| Time | % Drug released | |
|---|---|---|
| (in hours) | Example 1A | Example 1B |
| 0.25 | 3 | 1 |
| 0.5 | 9 | 6 |
| 0.75 | 16 | 12 |
| 1 | 22 | 19 |
| 1.5 | 34 | 29 |
| 2 | 42 | 39 |
| 3 | 55 | 58 |
| 3.5 | 62 | 66 |
| 4 | 68 | 74 |
| 6 | 79 | 87 |

As observed from the above Table 7, the release of venetoclax from the tablets at time points 0.25, 0.5, 0.75, 1, 1.5 and 2 hours are distinguishably greater for Example 1A (tablets with hydroxypropyl cellulose inside solid dispersion) compared to Example 1B (without hydroxypropyl cellulose inside solid dispersion).

### Example 4

TABLE 8

| | |
|---|---|
| Ingredients | Quantity (% w/w) |

Solid Dispersion I

| | |
|---|---|
| Venetoclax | 10.41 |
| Co-povidone | 59.01 |
| Colloidal silicon dioxide | 0.49 |

Solid Dispersion II

| | |
|---|---|
| Polysorbate 80 | 6.07 |
| Colloidal silicon dioxide | 3.56 |
| Co-povidone | 10.41 |

Outside Dispersion

| | |
|---|---|
| Microcrystalline cellulose | 4.38 |
| Colloidal silicon dioxide | 1.96 |
| Sodium Stearyl fumarate | 0.55 |
| Core tablet weight | 96.84 |
| Film coating | 3.15 |
| Purified water | q.s. |
| Total tablet weight | 100.00 |

Manufacturing Process:

A. Solid Dispersion I:
a) Co-povidone and colloidal silicon dioxide were mixed and co-sifted.
b) Venetoclax was co-sifted with material of step (a).
c) Material of step (b) was further re-sifted.
d) Material of step (c) was blended.
e) Material of step (d) was extruded through hot melt extruder with suitable feed screw speed, twin screw speed, torque, pressure and zone temperature.
f) Extrudes of step (e) were milled in a suitable mill and sifted through suitable sieve to obtain granules of desired size.

B. Solid Dispersion II:
g) Co-povidone and colloidal silicon dioxide were sifted separately.
h) Polysorbate 80 was adsorbed on to colloidal silicon dioxide in a Rapid Mixer Granulator.
i) Granulated material of step (h) was co-sifted with co-povidone.
j) Material obtained in step (i) was blended.
k) Material obtained in step (j) was extruded through hot melt extruder with suitable feed screw speed, twin screw speed, torque, pressure and zone temperature.

C. Processing Other Components and Tableting:
l) Microcrystalline cellulose and colloidal silicon dioxide were co-sifted.
m) Step (f) material and step (k) material were co-sifted.
n) Sodium stearyl fumarate was sifted.
o) Material in step (l) and step (m) were blended.
p) Material in step (n) was blended with material in step (o).
q) Material in step (p) was compressed into tablet and film coated.

Parameters for Hot Melt Extrusion:

TABLE 9

| | Acceptance criteria | |
|---|---|---|
| Parameter | Solid dispersion I | Solid dispersion II |
| Description | Pale yellow to | White opaque |
| | yellow colored clear | extrudes |
| | extrudates | |
| Die Size | 3 mm | 3 mm |
| Screw speed | 20-200 rpm | 10-50 rpm |
| Zone temperature | 80-160° C. | 80-130° C. |

a. Torque:

TABLE 10

| | Value | |
|---|---|---|
| Attribute | Solid Dispersion I | Solid Dispersion II |
| Torque value | <80% (Acceptable) | <20% (Acceptable) |
| Throughput | High | High |

b. Clarity:

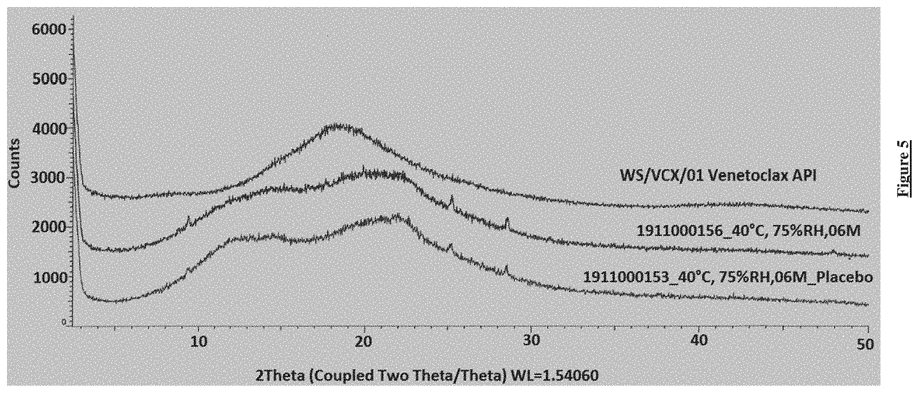

Extrudes of Solid dispersion I and Solid dispersion II as per Example 4 exhibited complete and homogenous molecular dispersion as observed in FIGS. 3 and 4, respectively.

c. Dissolution Studies:

Tablets prepared as per Example 4 and the reference product Venclexta® tablet manufactured by Abbvie Inc. were subjected to dissolution in 250 mL of pH 6.8 phosphate buffer with 0.4% sodium dodecyl sulphate, USP Type III apparatus (Bottom 200 mesh size ss) at dip rate of 20 DPM. Results are as provided below:

TABLE 11

| | % Drug released | |
|---|---|---|
| | Example 4 | Venclexta ® |
| Time (in hours) | composition | (Reference) |
| 0.25 | 5 | 8 |
| 0.5 | 14 | 17 |
| 0.75 | 23 | 25 |
| 1 | 32 | 33 |
| 1.5 | 48 | 46 |
| 2.0 | 61 | 57 |
| 3.0 | 82 | 77 |
| 3.5 | 90 | 91 |
| 4.0 | 92 | 97 |
| 5.0 | 92 | 98 |

### Example 5

Preparation of Amorphous Form of Venetoclax:

Dimethyl sulfoxide (275 ml) was charged to the reaction vessel and the temperature was adjusted to 30±5° C., if required. Venetoclax sodium (100 g) was added to reaction vessel at 30±5° C. The reaction vessel was flushed with 25 ml dimethyl sulfoxide at 30±5° C. The reaction mass was stirred for 25-55 minutes at 30±5° C. The reaction mass was filtered at 30±5° C. and washed with 30 ml dimethyl sulfoxide at 30±5° C.

Meanwhile 1900 ml DM water was charged to another reaction vessel and temperature was adjusted to 30±5° C., if required. Citric acid monohydrate (11.8 g) was charged in to DM water at 30±5° C. The solution of citric acid monohydrate was stirred to get clear solution. The solution was filtered at 30±5° C. The reaction vessel was washed with 200 ml DM water and filtered it at 30±5° C.

The venetoclax and dimethyl sulfoxide mixture, and solution of citric acid monohydrate was combined through addition vessel. The reaction mass was stirred for 85-95 minutes at 52±3° C. The reaction mass was filtered at 52±3° C. The wet cake was washed with (pre-heated) (2×100 ml) DM water at 52±3° C. The material is dried and unloaded. DM water (900 ml) was charged to reaction vessel and the temperature of DM water was raised to 52±3° C. The unloaded wet cake was charged to the DM water at 52±3° C. The reaction vessel was flushed with 100 ml DM water. The reaction mass was stirred for 3 hours±5 minutes at 52±3° C. The reaction mass was filtered at 52±3° C. The wet cake was washed with (pre-heated) (2×100 ml) DM water at 52±3° C. The material is dried and unloaded. The wet cake was dried in vacuum tray dryers for 1 hour±10 minutes under vacuum without applying any utility and then dried at 70±5° C. for 09 hours±10 minutes. Material was unloaded and milled. The milled material was collected in a cleaned container having double polythene bags. DM water (900 ml) was charged in to reaction vessel. The temperature of DM water was raised to 52±3° C. The milled material was charged in to the DM water at 52±3° C. The reaction vessel was flushed with 100 ml DM water at 52±3° C. The reaction mass was stirred for 3 hours±5 minutes at 52±3° C. The reaction mass was filtered at 52±3° C. The wet cake was washed with (pre-heated) (2×100 ml) DM water at 52±3° C. The material is dried and unloaded. The wet cake was dried in VTD for 1 hour±10 minutes under vacuum without applying any utility and then dried at 70±5° C. for 06 hours±10 minutes. The material was milled and collected in a cleaned container having double polythene bags. Tray filled with DM water was placed at lower bottom shelf of VTD. The temperature of VTD was raised to 70±5° C. without applying vacuum. The vacuum oven was maintained without applying Vacuum for 20-40 minutes at 70±5° C. The above milled material was charged in VTD. The material was dried under vacuum for 2 hours±10 min at 70±5° C. The material was unloaded and packed.

### Example 6

XRPD of amorphous form of venetoclax in Example 5, venetoclax composition of Example 4 and placebo:

XRPD of amorphous form of venetolcax prepared as per Example 5, the venetoclax composition of Example 4 and placebo composition similar to Example 4 without venetoclax was analysed after storage for 6 month at 40° C./75% RH. XRPD was measured on PANalytical X'Pert PRO X-Ray Diffractometer using CuKal radiation. The samples were measured in reflection mode in the range 2.5-50°. LYNXEYE XE T (1D mode) was used as detector. It was observed that the formulation as per Example 4 retains the amorphous form of venetoclax and is therefore stable. The XRPD data is provided in FIG. 5.

1. A solid dispersion comprising venetoclax or a pharmaceutically acceptable salt thereof, at least one pharmaceutically acceptable polymer and optionally at least one pharmaceutically acceptable glidant, wherein the solid dispersion is devoid of surfactant.

2. The solid dispersion of claim 1, wherein venetoclax or a pharmaceutically acceptable salt thereof is present in an amount of about 1% to about 20% by weight.

3. The solid dispersion of claim 1, wherein at least one polymer is present in an amount of about 35% to about 90% by weight.

4. The solid dispersion of claim 1, wherein at least one polymer is selected from group consisting of hydrophilic water soluble polymers, enteric coating polymers and water insoluble polymers.

5. The solid dispersion of claim 1, wherein at least one polymer is selected from group consisting of hydroxypropyl cellulose, hydroxyethyl cellulose, hydroxypropyl methyl cellulose, methyl cellulose, polyvinylpyrrolidone, co-povidone, carrageenans, galactomannans and xanthan gum, cyclodextrins, polyethylene glycols, polyethylene oxides, copolymers and graft copolymers of polyethylene glycols or polyethylene oxides, ethylcellulose, low substituted hydroxypropyl cellulose, cellulose acetate phthalate, cellulose acetate trimellitate, cellulose acetate succinate, methylcellulose phthalate, hydroxymethylcellulose ethyl phthalate, hydroxypropylmethylcellulose phthalate, hydroxypropylmethylcellulose acetate succinate, hydroxypropylmethyl acetate maleate, hydroxypropylmethyl trimellitate, carboxymethylethylcellulose, polyvinyl butyrate phthalate, polyvinyl alcohol acetate phthalate.

6. The solid dispersion of claim 1, wherein at least one polymer is co-povidone.

7. The solid dispersion of claim 1, wherein at least one glidant comprises colloidal silicon dioxide.

8. A pharmaceutical composition comprising a mixture of: (a) first solid dispersion comprising venetoclax or a pharmaceutically acceptable salt thereof, at least one pharmaceutically acceptable polymer and optionally at least one pharmaceutically acceptable glidant; and (b) second solid dispersion comprising at least one pharmaceutically acceptable polymer, at least one surfactant and optionally at least one pharmaceutically acceptable glidant, wherein the second solid dispersion is free of venetoclax

9. The composition of claim 8, wherein the first solid dispersion is devoid of surfactant.

10. The composition of claim 8, wherein venetoclax or a pharmaceutically acceptable salt thereof is present in an amount of about 1% to about 20% by weight of first solid dispersion.

11. The composition of claim 8, wherein at least one polymer in first solid dispersion is present in an amount of about 35% to about 90% by weight of first solid dispersion.

12. The composition of claim 8, wherein at least one polymer is selected from group consisting of hydrophilic water soluble polymers, enteric coating polymers and water insoluble polymers.

13. The composition of claim 8, wherein at least one polymer is selected from group consisting of hydroxypropyl cellulose, hydroxyethyl cellulose, hydroxypropyl methyl cellulose, methyl cellulose, polyvinylpyrrolidone, co-povidone, carrageenans, galactomannans and xanthan gum, cyclodextrins, polyethylene glycols, polyethylene oxides, copolymers and graft copolymers of polyethylene glycols or polyethylene oxides, ethylcellulose, low substituted hydroxypropyl cellulose, cellulose acetate phthalate, cellulose acetate trimellitate, cellulose acetate succinate, methylcellulose phthalate, hydroxymethylcellulose ethyl phthalate, hydroxypropylmethylcellulose phthalate, hydroxypropylmethylcellulose acetate succinate, hydroxypropylmethyl acetate maleate, hydroxypropylmethyl trimellitate, carboxymethylethylcellulose, polyvinyl butyrate phthalate, polyvinyl alcohol acetate phthalate.

14. The composition of claim 8, wherein at least one polymer is co-povidone.

15. The composition of claim 8, wherein at least one glidant is colloidal silicon dioxide.

16. The composition of claim 8, wherein at least one surfactant is selected from group consisting of anionic or non-ionic surfactants or mixtures thereof.

17. The composition of claim 8, wherein at least one surfactant is selected from group consisting of PEG-35 castor oil, PEG-40 hydrogenated castor, PEG-60 hydrogenated castor oil, sorbitan monooleate, sorbitan monostearate, sorbitan monopalmitate, sorbitan monolaurate, sorbitan tristearate, sorbitan trioleate, PEG-20 sorbitan monooleate, PEG-20 sorbitan monostearate, PEG-20 sorbitan monopalmitate, PEG-20 sorbitan monolaurate, polyoxyethylene (20) sorbitan tristearate, polyoxyethylene (20) sorbitan trioleate, PEG (2) stearyl ether, macrogol 6 cetylstearyl ether or macrogol 25 cetylstearyl ether, α-tocopheryl polyethylene glycol succinate or mixtures thereof.

18. The composition of claim 8, wherein at least one surfactant is polysorbate 80.

19. The composition of claim 8, wherein at least one surfactant is present in an amount of about 1% to about 10% by total weight of the composition.

20. The composition of claim 9, wherein venetoclax or a pharmaceutically acceptable salt thereof is present in an amount of about 1% to about 20% by weight of first solid dispersion.

21. The composition of claim 9, wherein at least one polymer in first solid dispersion is present in an amount of about 35% to about 90% by weight of first solid dispersion.

22. The composition of claim 9, wherein at least one polymer is selected from group consisting of hydrophilic water soluble polymers, enteric coating polymers and water insoluble polymers.

23. The composition of claim 9, wherein at least one polymer is co-povidone.

24. The composition of claim 9, wherein at least one glidant is colloidal silicon dioxide.

25. The composition of claim 9, wherein at least one surfactant is selected from group consisting of anionic or non-ionic surfactants or mixtures.

26. The composition of claim 9, wherein at least one surfactant is polysorbate 80.

27. The composition of claim 9, wherein at least one surfactant is present in an amount of about 1% to about 10% by weight of the composition.

28. A solid dispersion comprising venetoclax or a pharmaceutically acceptable salt thereof, at least two pharmaceutically acceptable polymers and optionally at least one pharmaceutically acceptable glidant, wherein the solid dispersion is devoid of surfactant.

29. The solid dispersion of claim 28, wherein venetoclax or a pharmaceutically acceptable salt thereof is present in an amount of about 1% to about 20% by weight.

30. The solid dispersion of claim 28, wherein at least two polymers are present in an amount of about 35% to about 90% by weight.

31. The solid dispersion of claim 28, wherein at least two polymers are selected from group consisting of hydrophilic water soluble polymers, enteric coating polymers and water insoluble polymers.

32. The solid dispersion of claim 28, wherein at least two polymers are selected from group consisting of hydroxypropyl cellulose, hydroxyethyl cellulose, hydroxypropyl methyl cellulose, methyl cellulose, polyvinylpyrrolidone, co-povidone, carrageenans, galactomannans and xanthan gum, cyclodextrins, polyethylene glycols, polyethylene oxides, copolymers and graft copolymers of polyethylene glycols or polyethylene oxides, ethylcellulose, low substituted hydroxypropyl cellulose, cellulose acetate phthalate, cellulose acetate trimellitate, cellulose acetate succinate, methylcellulose phthalate, hydroxymethylcellulose ethyl phthalate, hydroxypropylmethylcellulose phthalate, hydroxypropylmethylcellulose acetate succinate, hydroxypropylmethyl acetate maleate, hydroxypropylmethyl trimellitate, carboxymethylethylcellulose, polyvinyl butyrate phthalate, polyvinyl alcohol acetate phthalate.

33. The solid dispersion of claim 28, wherein the at least two pharmaceutically acceptable polymers are different and are water soluble.

34. A solid dispersion comprising at least one polymer, a surfactant, and optionally one or more pharmaceutically acceptable glidants, wherein the solid dispersion is free of venetoclax or a pharmaceutically acceptable salt thereof.

35. A pharmaceutical composition comprising a solid dispersion free of surfactant comprising venetoclax or a pharmaceutically acceptable salt thereof, at least two different polymers and optionally one or more pharmaceutically acceptable glidants.

36. The solid dispersion as described herein prepared by hot melt extrusion.

37. The solid dispersion of claim 36, wherein the input venetoclax or a pharmaceutically acceptable salt thereof is at least substantially amorphous.

38. A pharmaceutical composition as described herein in tablet form and bioequivalent to Venclexta®

39. A pharmaceutical composition as described herein wherein the venetoclax or a pharmaceutically acceptable salt remains at least substantially amorphous after storage for 6 months at 40° C./75% RH substantially as shown in FIG. 5.

40. A pharmaceutical composition as described herein wherein which exhibits the dissolution profile substantially as shown in Table 11.
